(19) United States
(12) Patent Application Publication
(10) Pub. No.: US 2003/0149450 A1
(43) Pub. Date: Aug. 7, 2003

(54) Brainstem and cerebellar modulation of cardiovascular response and disease

(76) Inventor: Marc R. Mayberg, Chagrin Falls, OH (US)

Correspondence Address: Pepper Hamilton LLP, One Mellon Center, 50th Floor, 500 Grant Street, Pittsburgh, PA, 15219, US

(21) Appl. No.: 10/357,161

(22) Filed: Feb. 3, 2003

Related U.S. Application Data
(60) Provisional application No. 60/353,701, filed on Feb. 1, 2002.

(51) Int. Cl. A61N 1/18

(52) U.S. Cl. 607/3

(57) ABSTRACT

The present invention is directed to an apparatus and methods for modulating brainstem and cerebellar circuits controlling blood pressure or heart rate using a variety of techniques including but not limited to surface stimulation, depth electrode stimulation, and localized infusion of agents to these regions.

### CROSS-REFERENCE TO RELATED APPLICATIONS

This application claims priority from U.S. provisional patent application Serial No. 60/353,701 filed Feb. 1, 2002, the contents of which is incorporated by reference in its entirety.

### BACKGROUND OF THE INVENTION

The present invention concerns a system for treating a cardiovascular disorder by artificial neural stimulation. More particularly, it relates to an implantable medical device configured to provide both electrical and/or chemical stimulation in a region of a patient's brainstem and/or, cerebellum causing regulation of the heart, vasculature and other bodily systems.

A variety of different cardiovascular ailments relate to, or are caused by, abnormal blood pressure or heart rate regulation. In general terms, the heart functions to pump blood containing oxygen and nutrients to bodily tissues and organs. Factors which determine blood pressure include heart rate and stroke volume (cardiac output), vascular resistance, arterial compliance, and blood volume. Blood being pumped to and from the heart develops a pressure (or blood pressure) in the heart and arteries. Blood pressure is determined by cardiac output and peripheral vascular resistance. The cardiac output, in turn, is a function of heart rate and stroke volume.

Hypertension, or elevated blood pressure, is a relatively common affliction. A 1993 Canadian study of 1,374 individuals ranging from 30 to 69 years of age found that 32% of the male adults and 19% of the female adults in the study exhibited high blood pressure. Most patients with hypertension exhibit the hemodynamic abnormality of increased vascular resistance. Treatment is essential to limit secondary organ damage to the heart, kidneys, brain and eyes, and other effects which tend to contribute to early death of the hypertensive person.

Refractory hypertension is characterized by blood pressure that remains above 140/90 mm Hg (160/90 mm Hg where the subject is greater than 60 years of age). Although treatment with anti-hypertensive drugs for a period of time is normally adequate to relieve hypertension, refractory hypertension is not as readily treatable. The cause of the refractory hypertension is the basis on which the disorder is classified. Some examples are secondary hypertension, where a specific underlying disorder—such as kidney disease—is present; presence of exogenous substances which may increase blood pressure or interfere with anti-hypertensive medication; biological factors—such as obesity; inappropriate or inadequate treatment of the disorder; and noncomplying drug ingestion attributable to complex dosing schedules or medicinal side effects.

Given the above, treatment of abnormal blood pressure-related cardiovascular disorders, such as hypertension and congestive heart failure, focus upon adjusting heart rate, stroke volume, peripheral vascular resistance, or a combination thereof. With respect to heart rate, one area of particular interest is vagal control. The rate of the heart is restrained by vagus nerves in conjunction with cardiac depressor nerves. The vagus nerves extend from the medulla and innervate the heart (as well as other organs). The medulla, in turn, regulates sympathetic and parasympathetic nervous system output, and can affect heart rate in part by controlling vagus nerve activity (or vagal tone) to the heart. The medulla exerts this autonomic control over the heart in response to sensed changes in blood pressure. More particularly, a series of pressure sensitive nerve endings, known as baroreceptors, are located along the carotid sinus, a dilated area at the bifurcation of the common carotid artery. The baroreceptors are formed at the terminal end of the carotid sinus nerve (or Hering's nerve), which is a branch of the glossopharyngeal nerve. The glossopharyngeal nerve extends to the medulla such that the carotid sinus baroreceptors communicate (or signal) with the medulla with carotid sinus pressure information. A reflex pathway (or baroreflex) is thereby established, with the medulla automatically causing an adjustment in heart rate in response to a pressure change in the carotid sinus. For example, a rise in carotid pressure causes the medulla to increase vagal neuronal activity. The above-described reflex pathway (or baroreflex) results in a lowering of the heart rate. A similar relationship is found with myocardial baroreceptors on the aortic arch. Notably, bodily systems other than the heart, such as the systemic vasculature and kidneys, are also influenced by nerve stimulation and contribute to overall cardiovascular regulation. In light of this vagally-mediated, baroreflex control of heart rate and other bodily systems, it may be possible to regulate heart rate, and thus blood pressure, by artificially stimulating the carotid sinus nerves, myocardial nerves, other cardiovascular influencing nerves, or brain structures to control both hypotension and hypertension as well as bradycardia and tachycardia.

### SUMMARY

The present invention provides for apparatus and methods to stimulate regions near the hindbrain in order to control or modulate the cardiovascular response or state of a vertebrate. Such stimulation may involve using a variety of techniques including, but not limited to, surface stimulation, depth electrode stimulation, and localized infusion of pharmaceutical agents to these regions. The present invention also includes direct modulation of centrally mediated cardiovascular responses through devices placed in or near the appropriate target sites in the cerebellum, hindbrain, and brainstem.

In one embodiment, an apparatus for modulating activity or function of a hindbrain structure in a vertebrate comprises a therapy delivery device positioned near a site of the hindbrain structure of the vertebrate for modulating the function of the hindbrain and a controller or pulse generator electrically connected to the therapy delivery device to enable it to deliver the therapy. In the apparatus, the therapy delivery device may be one or more electrodes. Alternately the therapy delivery device may be a catheter or infuser that delivers a pharmaceutical reagent to a site of the hindbrain structure. The therapy delivery device may comprise electrodes and pharmaceutical therapy delivery devices. Either the electrodes and or the catheter are connected to a controller. Preferably the therapeutic device is at a site near a surface of the patient's hindbrain and even more preferably is implanted in the body of the patient at a site near said hindbrain structure. The hindbrain structure may comprise but is not limited to the medulla, the cerebellum, the nucleus tractus solitarius, the caudal ventrolateral medulla, the rostral ventrolateral medulla, fastigial nucleus, or the dorsomedial medulla.

The apparatus may further comprise one or more sensors that measures the cardiovascular state or response of a patient or other vertebrate with the sensor being electrically connected to the controller.

In another embodiment, an apparatus for modulating autonomic response in a vertebrate comprises a therapy delivery device positioned near a site of the hindbrain structure of the vertebrate for modulating the function of the hindbrain and a controller or pulse generator electrically connected to the therapy delivery device to enable it to deliver the therapy. In the apparatus, the therapy delivery device may be one or more electrodes. Alternately the therapy delivery device may be a catheter or infuser that delivers a pharmaceutical reagent to a site of the hindbrain structure. The therapy delivery device may comprise electrodes and pharmaceutical therapy delivery devices. Either the electrodes and or the catheter are connected to a controller. Preferably the therapeutic device is at a site near a surface of the patient's hindbrain and even more preferably is implanted in the body of the patient at a site near said hindbrain structure. The hindbrain structure may comprise the medulla, the cerebellum, the nucleus tractus solitarius, the caudal ventrolateral medulla, the rostral ventrolateral medulla, fastigial nucleus, or the dorsomedial medulla.

The apparatus may further comprise one or more sensors that measures the cardiovascular state or response of a patient or other vertebrate with the sensor being electrically connected to the controller.

In one embodiment of the present invention a method of determining the placement of a therapy delivery device for modulating the activity or function of a hindbrain structure comprising: delivering a therapy near a site of a hindbrain structure of said vertebrate and measuring the cardiovascular state of said vertebrate.

In another embodiment, a method of controlling the cardiovascular state of a vertebrate or patient comprises comparing the cardiovascular state of the vertebrate or patient to a normal or previous cardiovascular state or response and delivering a therapy in a sufficient amount using the therapeutic delivery device to return the vertebrate to its normal cardiovascular state. The method may further comprising the step of measuring the cardiovascular state of the vertebrate with sensors such as pH, blood pressure, heart rate, dissolved oxygen, and dissolved carbon dioxide. Based on the cardiovascular state of the patient input from the sensors into the controller, the cardiac output is determined by software and hardware in the controller. Based on the cardiac output, the one or more therapy delivery devices may be activated to deliver a pharmaceutical or an electrical stimulation to a region near a hindbrain structure of the patient. The steps of comparing the cardiovascular state as measured by the sensors and delivering the therapy to a region near a hindbrain structure in the patient are performed in a closed loop and may use fuzzy logic algorithms to determine output from the therapeutic delivery device. The method may comprise multiple therapy delivery devices which are used and are enabled in response to the results of the step of comparing the cardiovascular state of the vertebrate to a normal state. The method of delivering a therapy may include the step of changing the output from the therapeutic delivery device, wherein the out is chosen from the group consisting of voltage, pulse width, pulse frequency, current, drug delivery rate, and drug concentration. The method may use a pharmaceutical which acts on the autonomic system and may include such pharmaceuticals as clonidine, guanethidine, a vetatrum alkaloid, alpha blockers and, specific neural excitatory or inhibitory transmitters and their antagonists such as gamma-aminobutyric acid (GABA), glycine, norepinephrine, acetylcholine (Ach), or nitric oxide (NO), proteins or enymes which modify the metabolism, release, binding and re-uptake of neurotransmitters, and genes and gene products which regulate cellular processes related to neural transmission.

Advantages of the present invention are that both exciting and inhibitory brain centers for controlling cardiovascular response are may be stimulated or inhibited through the use of electrical stimulation or delivery of pharmaceuticals to the sites of the brain responsible for control of the cardiovascular (baroreflex) state of the patient. This invention may reduce or eliminate the amount of pharmaceutical required compared with traditional therapeutic treatments of cardiovascular conditions and may provide more precise real-time adjustment of a patient's cardiovascular state through use of closed loop control of the apparatus.

A preferred embodiment of the present invention provides an apparatus for modulating cardiovascular activity of a hindbrain structure in a vertebrate comprising: a therapeutic delivery device positioned near a site of the hindbrain structure of said vertebrate for modulating the function of said hindbrain; and a controller connected to said therapy delivery device to deliver the therapy. It is preferred that the therapeutic delivery device is an electrode connected to said controller. Alternatively, or in conjunction, it is preferred that the therapeutic delivery device delivers a pharmaceutical reagent to a site of said hindbrain structure for controlling cardiovascular activity in a vertebrate and is connected to said controller. In this embodiment, it is preferable that the apparatus further comprises a sensor that measures a cardiovascular state of said vertebrate and is electronically connected to said controller, an that the therapy delivering device is located at a site near the surface of said hindbrain structure. It is preferable that the therapy delivering device is implanted in the body of said vertebrate at a site near said hindbrain structure an preferably the hindbrain structure is selected from the group consisting of the medulla, the cerebellum, the nucleus tractus solitarius, verntrolateral medulla, the rostral ventrolateral medulla, and the dorsomedial medulla.

Another embodiment of the present invention comprises a method of determining the placement of a therapy delivery device for modulating the activity of a hindbrain structure comprising: delivering a therapy near a site of a hindbrain structure of said vertebrate and measuring the cardiovascular state of said vertebrate.

Various aspects and applications of the present invention will become apparent to the skilled artisan upon consideration of the brief description of the figures an the detailed description of the invention which follows.

### DESCRIPTION OF THE DRAWINGS

Aspects, features, benefits and advantages of the embodiments of the present invention will be apparent with regard to the following description, appended claims and accompanying drawings where:

FIG. 1 is a sagittal view of the brain illustrating placement of therapeutic delivery devices in a patient;

FIG. 2 is a axial view illustrating a positioning of therapeutic delivery devices of the present invention by feedthrough of leads through the subarachnoid space of the spinal column;

FIG. 3 is a schematic illustration of the components which may be used in a controller of the present invention;

FIG. 4 is an illustration of a block diagram of an algorithm to determine action which may be taken by the controller microprocessor in response to sensor input from the patient;

FIG. 5 is a schematic illustration of the baroreceptor vasomotor and heart rate reflex.

### DESCRIPTION OF THE INVENTION

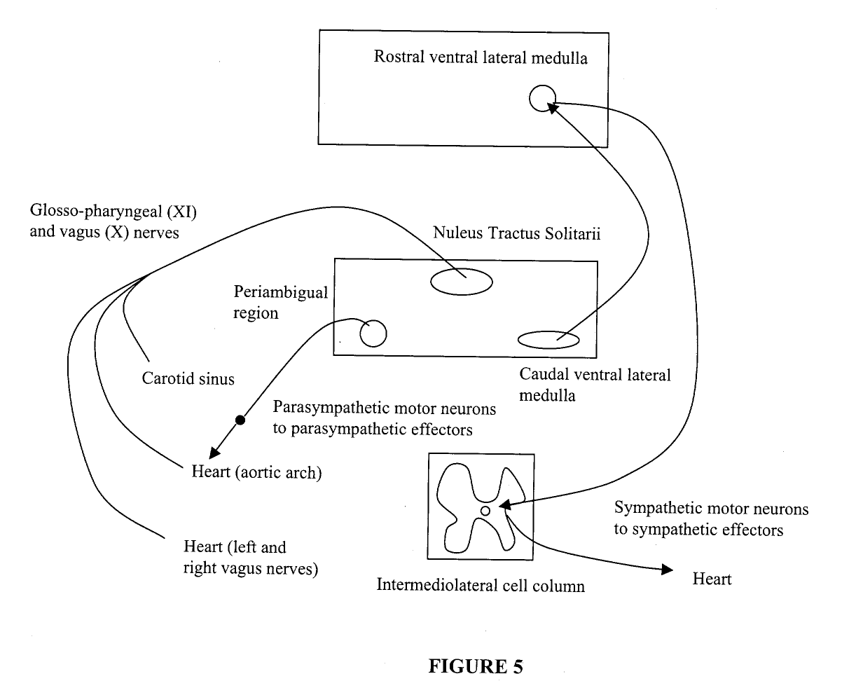

Treatment of cardiovascular disorders characterized by increased heart rate or blood pressure, such as hypertension or congestive heart failure, by neural stimulation presents a highly viable therapy. Substantial evidence in animal models and indirect evidence in humans has demonstrated that focal neuronal mechanisms exist for the central control of systemic blood pressure and heart rate. Both Willette R N, Barcas P P, Krieger A J, Sapru H N. Vasopressor and vasodepressor areas in the rat medulla. Neuropharmacol 1983; 22:1071-9 and Ciriello J, Caverson M M, Polosa C. Function of the ventral lateral medulla in the control of circulation. Brain Res Rev 1986; 11:359-91 illustrate the viability of this technique. In specific, the rostral ventral lateral medulla (RVLM) and caudal ventral lateral medulla (CVLM) mediate cardiovascular responses in a variety of settings. Stimulation or modulation of RVLM function elicits increased mean arterial pressure and heart rate, whereas stimulation or modulation of CVLM function evokes a cardiovascular depressor response, FIG. 5. In addition, the fastigial nucleus of the cerebellum has also been implicated in a central modulation of blood pressure. These structures of the hindbrain (metencephalon and myelencephalon) are acted on by the nucleus tractus solitarius in response to central projections of baroreceptor fibers from both aortic depressor and carotid sinus nerves entering the dorsolateral medulla oblongata. A substantial need exists for a neural stimulation device configured to deliver electrical stimulation to change the activity in or near the appropriate target sites in the hindbrain, the cerebellum, and brainstem alone or in combination with a stimulating drug or hindbrain activity modulating pharmaceutical directly to the same region.

The present invention provides various apparatus and methods to modulate hindbrain, brainstem and/or cerebellar circuits controlling blood pressure or heart rate activity using a variety of techniques including, but not limited to, surface stimulation, depth electrode stimulation, and localized infusion of agents to these regions. The present invention also includes direct modulation of centrally mediated cardiovascular responses through devices placed in or near the appropriate target sites in the cerebellum, hindbrain, and brainstem.

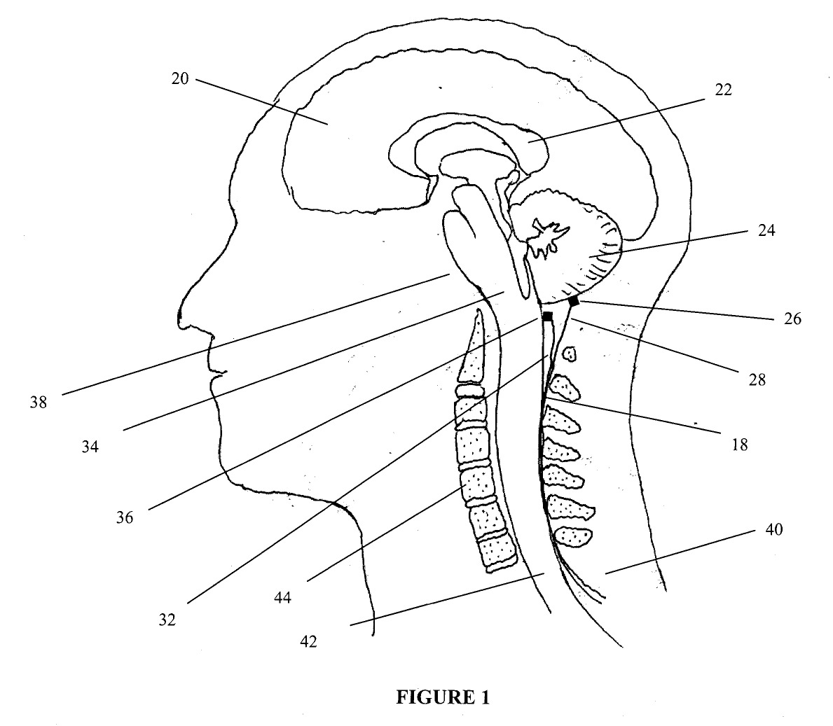

With reference to FIG. 1, an illustration, not to scale, is provided of cerebral cortex 20, corpus callosum 22, cerebellum 24, therapeutic delivery device 26 with lead 28 on the cerebellum, vertebrae 44, medulla 34, therapeutic delivery device 36 with lead 32 on the medulla, pons 38, spinal cord 42, and leads 28 and 32 from therapeutic delivery devices 26 and 36 at 40 for connection to controller (not shown) through the subarachoid space 18.

The apparatus for modulating cardiovascular activity or function of the hindbrain structure in a vertabrate or patient comprises one or more therapy delivery devices positioned near a site of the hindbrain structure of the vertebrate that is responsible or contributes to control of cardiovascular activity or function in the vertebrate. A controller or pulse generator is electrically connected to the therapy delivery device to enable it to deliver the therapy and to read sensors. In the apparatus, the therapy delivery device may be one or more electrodes. Alternately, the therapy delivery device may be a catheter, an infuser, or sustained release matrix as disclosed in U.S. Pat. No. 6,256,542 which is hereby incorporated by reference in its entirety, that delivers a pharmaceutical reagent to a site of the hindbrain structure. The therapy delivery device may comprise electrodes and pharmaceutical therapy delivery devices. Either the electrodes and or the catheter are connected to a controller. Preferably the therapeutic device is at a site near a surface of the patient's hindbrain and even more preferably is implanted in the body of the patient at a site near the hindbrain responsible for cardiovascular regulation. The hindbrain is the posterior of the three primary divisions of the vertebrate brain or the parts developed from it including the cerebellum, pons, and the medulla oblongata. Structures on the hindbrain responsible for cardiovascular regulation may comprise the medulla, the cerebellum, the nucleus tractus solitarius, the caudal ventrolateral medulla, the rostral ventrolateral medulla, fastigial nucleus, or the dorsomedial medulla.

Therapeutic delivery devices may include electrodes, catheters, infusers, sustained release matrix, a proportionally controlled orifice, or combinations of these. Different aspects of the present invention comprise new and novel methods of treating cardiovascular disorders by implantation of therapeutic delivery devices into specific area of the brain. It is to be understood that the term therapeutic delivery devices, as used here, is meant to include stimulation electrodes, drug-delivery catheters, sustained release matrixes, electrical sensors, chemical sensors or combinations of any of these at specific locations.

The electrode assembly of the present invention may be one electrode, multiple electrodes, or an array of electrodes in or around the target area. Electrical stimulation can be epidural, subdural or intraparenchymal. Electrodes in the present invention may comprise a quadripolar array in which associated ones of two pairs are secured to preselected sites; for example, on opposite sides of and adjacent to the hindbrain; they may also include the electrode configurations disclosed in U.S. Pat. Nos. 6,178,349 and 6,353,762 the teaching of which are incorporated herein by reference in their entirety. The electrodes may be composed of a biocompatible material and may include activated iridium, rhodium, titanium or platinum. The electrodes may be coated with a thin surface layer of iridium oxide to enhance electrical sensitivity. Electrodes may also comprise carbon, doped silicon, or silicon nitride. Each electrode may be provided with a biocompatible fabric “collar” or band about the electrode periphery to allow it to be readily sutured or glued into place using a surgical adhesive such as silicone adhesive. Electrodes which also comprise a drug delivery vehicle, such as those described in U.S. Pat. No. 6,178,349 incorporated herein by reference in its entirety, may also be used in the practice of embodiments of this invention. The electrodes are preferably small and typically about 0.5 to about 3 mm in diameter and may be in a flexible elastomeric sheath. For quadrapolar electrodes the leads terminate a the distal and proximal ends of the sheath in four electrically insulated cylindrical contact pads. The contact pads at the distal end are less than about 2 mm in length and are separated by an insulating distance, for example between 0.5 and about 2 mm. At the proximal end, which is anywhere from 25 to 50 centimeters distance from the distal end, a corresponding series of contacts are provided so that the electrode may be coupled to a potential source, a controller, or to a coupling lead which permits remote placement of the signal or input to the probe.

By a site on or near the hindbrain it is meant in the practice of various embodiments of this invention that the therapeutic delivery device, electrode, sensor, or drug delivery vehicle, is in contact with a site of the hindbrain. Contact may be through, for example, the cerebellar cortex material, epithelial cells, or a surgical adhesive. The location of the therapeutic delivery device at a site near to the hindbrain is such that it causes a physiological response, as measured by a change in the cardiovascular state or function of a patient, when a measurable electrical stimulation or pharmaceutical dose is administered to the site by the therapeutic delivery device near the hindbrain. Preferably the site near the hindbrain is on the surface of the hindbrain near to a structure, region, or nucleus that is to receive the therapy.

Another technique that offers the ability to affect hindbrain cardiovascular function in a reversible and dynamic fashion is the delivery of biological agents, or pharmaceutical drugs directly to target tissues via a patch, a subcutaneously implanted pump and/or a slow release matrix. Such drugs, for example but not limited to clonidine, guanethidine, a vetatrum alkaloid, alpha blockers, and midodrine, could be instilled precisely at such low doses as to completely avoid the side effects so common to modern therapy and to provide an increase or decrease in blood pressure or heart rate. Other categories of agents which could be locally instilled at selected hindbrain target sites include specific neural excitatory or inhibitory transmitters and their antagonists such as gamma-aminobutyric acid (GABA), glycine, norepinephrine, acetylcholine (Ach), or nitric oxide (NO), proteins or enymes which modify the metabolism, release, binding and re-uptake of neurotransmitters, and genes and gene products which regulate cellular processes related to neural transmission. Such doses could also be tailored in magnitude with respect to a particular patient's varying cardiovascular symptoms. Modulation may also occur or be enhanced by biological agents such as viral vectors, stem cells, gene therapy. The chemical or biological drug systems may be used as a primary treatment strategy or in combination with an electrically based one.

A combination therapeutic approaches, one combining electrical and biological or chemical means may also be used and modulated by the controller. In addition to the stimulation and chemical modulation, the implantable device could also have chemical and/or electrical sensing functions that can be coupled to the chemical and electrical output of the modulating device. Sensing can be done at the site of the electrode or the probe, at distant sites in the brain, heart, or other tissues. The apparatus may include sensing changes in physiological parameters such as heart rate, blood pressure or heart rate, respiratory changes, and other common indicators of cardiovascular disorders. The sensor information is used with controller hardware, microprocessor, analog and digital sensor inputs, multiplexers and filters, and controller software algorithms to determine the cardiovascular state of the patient, compare the state with a normal cardiovascular state, and determine which therapeutic delivery devices to activate and the amount of activation required to return the patient to a normal cardiovascular state.

The systolic measurement is the pressure of blood against artery walls when the heart has just finished pumping. It is the first or top number of a blood pressure reading. The second or bottom number is the diastolic measurement—the pressure of blood against artery walls between heartbeats when the heart is relaxed and filling with blood. Normal blood pressure is less than 130 mmHg systolic and less than 85 mmHg diastolic (130/85 or lower); for elderly patients, the first number (systolic) often is high (greater than 140 mmHg), while the second number (diastolic) is normal (less than 90 mmHg). This condition is called isolated systolic hypertension (ISH). Blood pressure is normally above 90/60 mm Hg. When the blood pressure is too low there is inadequate blood flow to the heart, brain, and other vital organs; such a condition may be due to heart failure, heart attack, changes in heart rhythm, or drugs. While these ranges are considered normal, depending on the patient, the normal range may be different. Similar ranges apply for other cardiovascular parameters which measure the cardiovascular state of the patient such as heart rate and blood oxygen levels. One normally skilled in the art would be able to determine the normal range of cardiovascular state in a patient without undue experimentation.

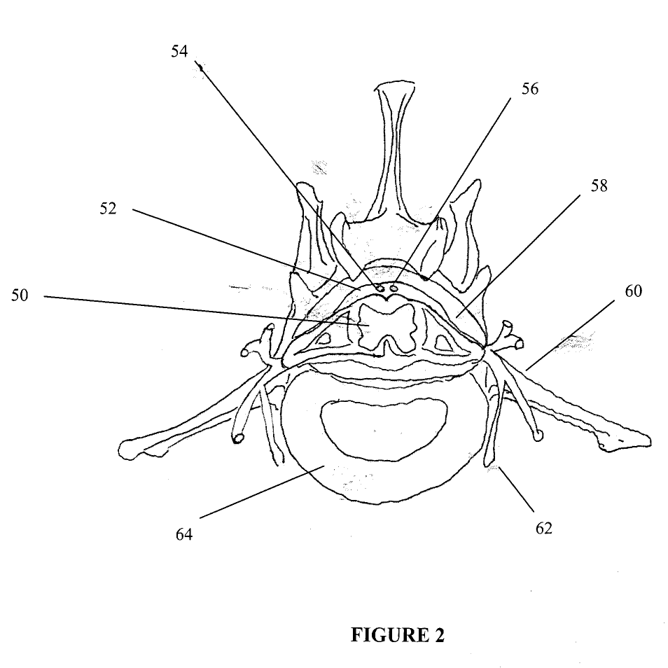

Implantation of the therapeutic delivery devices and controller may be performed by conventional stereotactic surgical techniques. Alternatively, an electrode or delivery device may be placed in the intrathecal space (subarachnoid space), FIG. 2, adjacent to the spinal column and the device manipulated into a region near the hindbrain through this space. With reference to FIG. 2, intrathecal or subarachnoid space 52, spinal cord 50, disk 64, dura mater 58, sympathetic nerve ganglion 62, vertebrae 60, and therapeutic delivery device leads 54 and 56 are illustrated. The leads 54 and 56 are shown in the subarachnoid space illustrating a method for positioning therapeutic delivery devices, not shown, in the hindbrain region of a subject. Real-time intraoperative imaging using magnetic resonance imaging (MRI) or computed tomography (CT) may be useful in localizing the position of the therapy delivering device to a site on the hindbrain. Once the one or more therapeutic delivery devices or sensors has been positioned in the desired region hindbrain for controlling cardiovascular function, the devices may be affixed to one or more sites near the hindbrain by suturing or gluing the device using a suitable surgical adhesive. Leads for power, signal output, and control signals between the controller and devices are preferable sheathed in a biologically suitable material such as polytetrafluoroethylene or PFA.

One surgical technique which may be used to insert a therapeutic delivery device of the present invention into a region of the hindbrain is a posterior fossa craniotomy—removal of occipital bone and direct visualization of cerebellum and brainstem. Manual insertion of depth electrodes into parenchyma is accomplished using image guidance techniques and or electrophysiologic mapping. Once the therapeutic delivery device has been placed on the cerebellum and brainstem, attachment of surface electrodes to these hindbrain structures is performed using an adhesive such as a tissue glue, microhooks, or use of a circumferential clamp or fastener.

Endoscopically, therapeutic delivery devices may be placed, using image guidance techniques and or electrophysiologic mapping, near the hindbrain through a posterior fossa burr hole or through a puncture of lumbar or cervical theca. Stereotactic placement of depth electrodes using image guidance techniques may allow placement of therapeutic delivery devices where the entry site is a frontal burr hole or where the entry site is a posterior fossa burr hole.

In the practice of embodiments of this invention depth electrodes may be placed at nuclei in medulla and cerebellum using anatomical references (similar to DBS) and electrophysiologic monitoring. Surface electrodes—unilateral or bilateral arrays may be placed over dorsal and or ventral medulla.

A controller is used to operate one or more therapeutic delivery devices to modulate cardiovascular function in the patient, to record the inputs of various sensor monitoring the cardiovascular state of the patient, and to compare and calculate the cardiovascular state of the patient with threshold limits for cardiac output, blood pressure, heart rate, and blood gas levels. The controller is used to supply power to the therapeutic delivery device and sensors and to receive input from sensors via electrical leads from the controller to these devices. The electrical leads should be sheathed in a biocompatible material such as polytetrafluoroethylene and should be flexible. Power supplied to the one or more therapeutic delivery devices may stimulate or inhibit the site of the hindbrain to which the device is located, for purposes of this disclosure both functions are sometimes included within the term “stimulating” (and its variations) in this specification. The controller may be powered by a battery, an external power supply, a fuel cell, or a battery pack for external use. When the therapeutic delivery device is one or more electrodes, the controller may change the output to the electrode by way of frequency of power, voltage, current, and or polarity in response to a comparison made by the controller of the cardiovascular state of the patient with the threshold limits. When the therapeutic delivery device delivers a pharmaceutical, the controller changes its output such that a pump, pressure source, proportionally controlled orifice, or heater increase or decreases the rate at which the pharmaceutical is delivered to the site near the hindbrain of the patient. The controller may operate any number or combination of electrodes, sensors, and pharmaceutical delivery devices, for example the controller may be connected to two electrodes, a pH sensor, and a peristaltic pump for delivering a pharmaceutical to a site of the hindbrain near one of the electrodes. The controller may be implanted within the patient or it may be positioned by leads outside of the patient. A portion of the control system may be external to the patient's body for use by the attending physician to program the implanted controller and to monitor its performance. This external portion may include a programming wand which communicates with the implanted device by means of telemetry via an internal antenna to transmit parameter values (as may be selectively changed from time to time by subsequent programming) selected at the programmer unit such as a computer. The programming wand also accepts telemetry data from the controller to monitor the performance of the implanted device.

The following parameters related to the electrical signal from the controller apply to the aforementioned embodiments and embodiments discussed in greater detail herein. The electrical signal to stimulate the at least one predetermined site may be continuous or intermittent. The electrode may be either monopolar, bipolar, or multipolar. The electrodes may operate as a cathode or an anode. Preferably, the oscillating electrical signal is operated at a voltage between about 0.1 microvolts to about 20 V. More preferably, the oscillating electrical signal is operated at a voltage between about 1 V to about 15 V. For microstimulation, it is preferable to stimulate within the range of 0.1 microvolts to about 1 V. Preferably, the electric signal source is operated at a frequency range between about 2 Hz to about 2500 Hz. More preferably, the electric signal source is operated at a frequency range between about 2 Hz to about 200 Hz. Preferably, the pulse width of the oscillating electrical signal is between about 10 microseconds to about 1,000 microseconds. More preferably, the pulse width of the oscillating electrical signal is between about 50 microseconds to about 500 microseconds. Preferably, the application of the oscillating electrical signal is: monopolar when the electrode is monopolar; bipolar when the electrode is bipolar; and multipolar when the electrode is multipolar.

Sensors are connected to the controller for power to operate and to receive sensor data to the controller. The sensors used to provide an indication of the cardiovascular condition or vagal tone of the patient's heart may be those described in U.S. Pat. No. 6,178,349, and U.S. Pat. Nos. 5,313,953, 6,442,420, 5,388,578, 6,353,762, and 5,411,031 the teachings of which are incorporated herein by reference. Detection of elevated blood pressure or heart rate may be accomplished using an electrode array implanted adjacent to an artery proximate the patient's heart, where it will sense the relatively small electrical resistance changes that accompany periodic blood pressure pressure or heart rate variations of the patient. Logic in the detection circuit of the sensor determines the patient's systolic and diastolic blood pressure pressure or heart rate from these resistance variations, in the form of an output signal. This signal is applied to the logic and controller circuit, and is monitored to ascertain an elevated level of the patient's systolic and/or diastolic blood pressure or heart rate that warrants intervention with the therapeutic delivery device. Other sensors useful for determining the cardiovascular condition, state, or response of the patient may include but are not limited to pH and or blood oxygen, an intracardiac pressure sensor, one or more electrodes, an external arm or finger cuff pressure sensor, or a flow probe place about an artery.

For some types of sensors, a microprocessor and analog to digital converter will not be necessary. The output from sensor can be filtered by an appropriate electronic filter in order to provide a control signal for signal generator. An example of such a filter is found in U.S. Pat. No. 5,259,387 “Muscle Artifact Filter, Issued to Victor de Pinto on Nov. 9, 1993, incorporated herein by reference in its entirety.

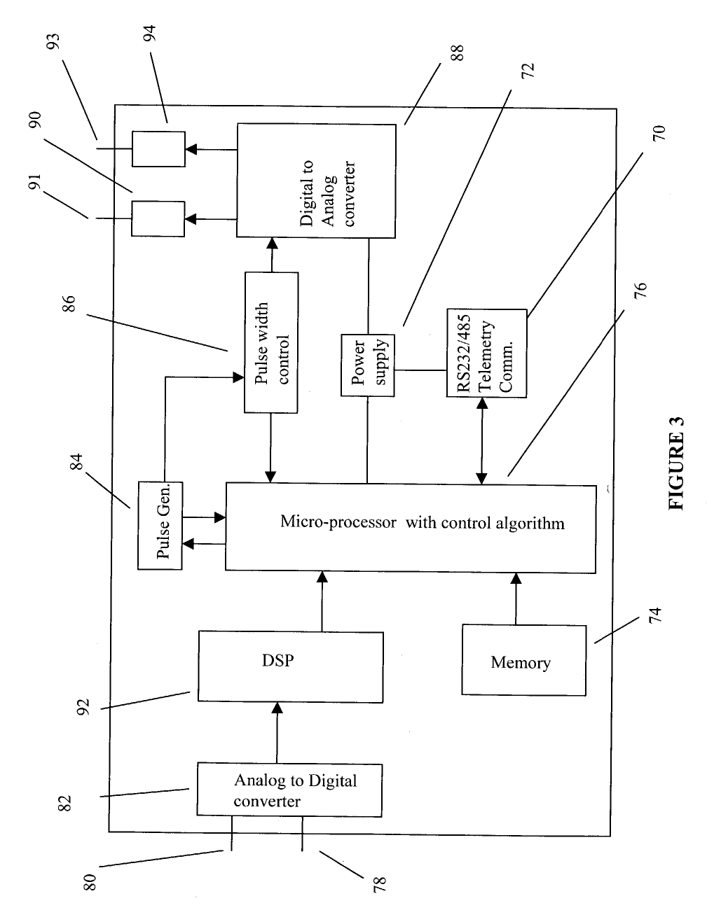

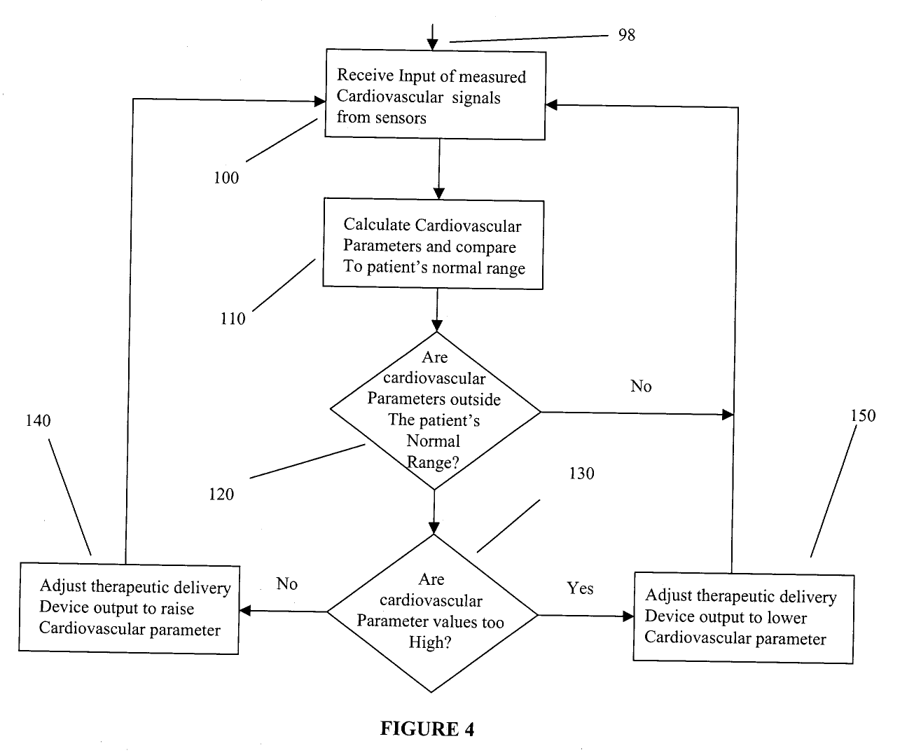

Closed-loop electrical stimulation can be achieved by a modified form of an implantable ITREL II signal generator available from Medtronics, Minneapolis, Minn. as disclosed in U.S. Pat. No. 6,353,762, the teaching of which is incorporated herein in its entirety, a controller as described in FIG. 3, or utilization of CIO DAS 08 and CIO-DAC 16 I processing boards and an IBM compatible computer available for Measurement Computing, Middleboro, Mass. with Visual Basic software for programming of alogoriths. With reference to FIG. 3 an illustration of a non-limiting example of a controller comprising a microprocessor 76 such as a PIC 16C73 from Microchip Technology, analog to digital converter 82 such as AD7714 from Analog Devices Corp., pulse generator 84 such as CD1877 from Harris Corporation, pulse width control 86, electrode driver 90, digital to analog converter 88 such as MAX538 from Maxim Corporation, power supply 72, memory 74, and communications port or telemetry chip 70 are shown. Optionally, a digital signal processor 92 is used for signal conditioning and filtering. Input leads 78 and 80 and output lead to electrode (therapeutic delivery device) 91 and drug delivery device (therapeutic deliver device) 93 are also illustrated. Additional electrodes, sensors, and therapeutic delivery devices may be added to the controller as required. As a nonlimiting example, inputs from sensors, such as pH and blood pressure sensors, are input to analog to digital converter 82. Microprocessor 76 receiving the sensor inputs uses algorithms to compute the cardiovascular state of the patient and using PID, Fuzzy logic, or other algorithms, computes an output to pulse generator and or drug delivery device drivers 90 and 94 to stimulate or inhibit sites in the hindbrain near which the therapeutic delivery devices are placed. The output of the analog to digital converter is connected to a microprocessor through a peripheral bus including address, data and control lines. The microprocessor processes the sensor data in different ways depending on the type of transducer in use. When the signal on sensor indicates a cardiovascular state outside of threshold values, for example blood pressure or heart rate, programmed by the clinician and stored in a memory, increasing amounts of stimulation to therapy delivery devices at sites near the hindbrain will be applied through output drivers of the controller. The output voltage or current from the controller are then generated in an appropriately configured form (voltage, current, frequency), and applied to the one or more therapeutic delivery devices implanted at sites near the hindbrain for a prescribed time period to reduce elevated blood pressure or heart rate and return the patient to a normal cardiovascular state. If the patient's blood pressure or heart rate as monitored by the system is not outside of the normal threshold limits (hypotensive or hypertensive, bradycardic or tachycardic), or if the controller output (after it has timed out) has resulted in a correction of the blood pressure or heart rate to within predetermined threshold range considered normal for the patient, no further stimuli are applied to the hindbrain and the controller continues to monitor the patient via the sensors. A block diagram of an algorithm which may be used in the present invention is shown in FIG. 4.

With reference to FIG. 4, suitably conditioned and converted sensor data 98 is input to the algorithm in block 100. The program computes cardiovascular parameter such as blood pressure, heart rate, or cardiac output, and compares the measured parameter to the patient's normal range for the parameter. The normal range will vary from patient to patient, but may be determined by a trained professional. These ranges are programmed into the microprocessor via the telemetry or communications port of the controller. The algorithm compares, 110, and then determines whether or not the cardiovascular parameters lie outside the patient's normal range, 120. If the measure cardiovascular parameter is not outside the patient's normal range, the program continues to monitor the sensors and reiterates the comparison part of the algorithm. If the measured cardiovascular parameter is outside of the patient's range, a determination or comparison is made, 130, as to whether the value is too high or too low compared with the normal range. If the cardiovascular parameter is too high an adjustment to the therapeutic delivery device is made, 150, to lower the cardiovascular state of the patient by calculating an output signal for pulse generator or drug delivery device to deliver a sufficient amount of the pharmaceutical or electrical stimulation to lower the cardiovascular state of the patient. The algorithm continues to monitor the cardiovascular state following the adjustment. If the cardiovascular parameter is too low then an adjustment to the therapeutic delivery device is made, 140, to raise the cardiovascular state of the patient by calculating an output signal for the pulse generator or drug delivery device to deliver a sufficient amount of a pharmaceutical or electrical stimulation to raise the cardiovascular state of the patient. The algorithm continues to monitor the cardiovascular state of the patient, 100, following the adjustment. The amount of adjustment made may be determined by proportional integral derivative algorithms of by implementation of Fuzzy logic rules.

The stimulus pulse frequency is controlled by programming a value to a programmable frequency generator using the bus of the controller. The programmable frequency generator provides an interrupt signal to microprocessor through an interrupt line when each stimulus pulse is to be generated. The frequency generator may be implemented by model CDP1878 sold by Harris Corporation. The amplitude for each stimulus pulse is programmed to a digital to analog converter using the controller's bus. The analog output is conveyed through a conductor to an output driver circuit to control stimulus amplitude. The microprocessor of the controller also programs a pulse width control module using the bus. The pulse width control provides an enabling pulse of duration equal to the pulse width via a conductor. Pulses with the selected characteristics are then delivered from signal generator through a cable and lead to the target locations of a brain or to a device such as a proportional valve or pump. The microprocessor executes an algorithm to provide stimulation with closed loop feedback control as shown in U.S. Pat. No. 5,792 which is incorporated herein by reference in its entirety.

Microprocessor executes an algorithm in order to provide stimulation with closed loop feedback control. At the time the stimulation device is implanted, the clinician programs certain key parameters into the memory of the implanted device via telemetry. These parameters may be updated subsequently as needed. The algorithm indicates the process of first choosing whether the neural activity at the stimulation site is to be blocked or facilitated and whether the sensor location is one for which an increase in the neural activity at that location is equivalent to an increase in neural activity at the stimulation target or vice versa. Next the clinician may program the range of values for pulse width, amplitude and frequency which device may use to optimize the therapy. The clinician may also choose the order in which the parameter changes are made. Alternatively, the clinician may elect to use default values or the microprocessor may be programmed to use fuzzy logic rules and algorithms to determine output from the therapeutic delivery device to the patient based on sensor data and threshold parameters for cardiovascular response.

In another embodiment, an apparatus for modulating autonomic response in a vertebrate comprises a therapy delivery device positioned near a site of the hindbrain structure of the vertebrate for modulating the autonomic response of the hindbrain and a controller or pulse generator electrically connected to the therapy delivery device to enable it to deliver the therapy. In the apparatus, the therapy delivery device may be one or more electrodes. Alternately the therapy delivery device may be a catheter or infuser or sustained release matrix that delivers a pharmaceutical reagent to a site of the hindbrain structure. The therapy delivery device may comprise electrodes and pharmaceutical therapy delivery devices. Either the electrodes and or the catheter are connected to a controller. Preferably the therapeutic device is at a site near a surface of the patient's hindbrain and even more preferably is implanted in the body of the patient at a site near said hindbrain structure. The hindbrain structure may comprise but is not limited to the medulla, the cerebellum, the nucleus tractus solitarius, the caudal ventrolateral medulla, the rostral ventrolateral medulla, fastigial nucleus, or the dorsomedial medulla. Modulating the function of the hindbrain structure is delivery of electrical stimulation and or a pharmaceutical by the therapeutic delivery device to increase or decrease the heart rate, blood pressure, or other cardiovascular condition of the vertebrae.

The apparatus of this embodiment may further comprise one or more sensors that measures the cardiovascular state or response of a patient or other vertebrate with the sensor being electrically connected to the controller.

In one embodiment of the present invention a method of determining the placement of a therapy delivery device, for example electrodes, sensors, catheters and microinfusion systems, for modulating the activity of a hindbrain structure related to cardiovascular function. The method comprises delivering a therapy near a site of a hindbrain structure of said vertebrate and measuring the cardiovascular state of said vertebrate and optimizing the response through an iterative process of delivering the therapy and measuring the patient's response.

In another embodiment, a method of controlling the cardiovascular condition of a subject comprises comparing the cardiovascular state of a vertebrate or patient to a normal cardiovascular state or response range and delivering a therapy in a sufficient amount to a hindbrain structure using the one or more therapeutic delivery devices to return the vertebrate or patient to its normal cardiovascular state or range. Delivery of the therapy may require one or more amounts or doses of the therapy, or example electrical pulses or microliters of a pharmacetical, to return the patient to its normal cardiovascular state. If a patient's cardiovascular state is within its normal range, it may be sufficient not to supply electrical stimulation or delivery of a pharmaceutical from the one or more therapeutic delivery devices to maintain the patient in its normal cardiovascular state. The method may further comprising the step of measuring the cardiovascular state of the vertebrate with sensors such as pH, blood pressure, heart rate dissolved oxygen, and dissolved carbon dioxide. For example, based on the cardiovascular state of the patient as measured by input from the sensors into the controller, the cardiac output, blood pressure or heart rate is determined by software and hardware in the controller. Based on the cardiac output, the one or more therapy delivery devices may be activated to deliver a pharmaceutical or an electrical stimulation to a region near a hindbrain structure responsible for cardiovascular function in the patient. The steps of comparing the cardiovascular state as measured by the sensors and delivering the therapy to a region near a hindbrain structure in the patient are performed in a closed loop and may utilize fuzzy logic rules and algorithms to determine output from the therapeutic delivery device to the patient. The method may comprise multiple therapy delivery devices which are used and are enabled in response to the results of the step of comparing the cardiovascular state of the vertebrate to a normal state. The method of delivering a therapy may include the step of changing the output from the therapeutic delivery device, wherein the output is chosen from the group consisting of voltage, pulse width, pulse frequency, current, drug delivery rate, and drug concentration. The method may use a pharmaceutical which acts on the autonomic system and may include such pharmaceuticals as clonidine, guanethidine, a vetatrum alkaloid, alpha blocker, and midodrine, or specific neural excitatory or inhibitory transmitters and their antagonists such as gamma-aminobutyric acid (GABA), glycine, norepinephrine, acetylcholine (Ach), or nitric oxide (NO), proteins or enymes which modify the metabolism, release, binding and re-uptake of neurotransmitters, and genes and gene products which regulate cellular processes related to neural transmission.

One aspect of the present invention provides an implantable medical device for enhanced stimulation of a region in the brain (e.g., the brainstem or cerebellum) of a patient to treat cardiovascular disorders. The devices includes an implantable pulse generator and an implantable electrode body implanted in or near the appropriate target sites in the cerebellum and brainstem. The electrode body includes an electrode electrically connected to the pulse generator. The electrode body is configured to sustain long-term contact between the electrode and the brainstem or cerebellum following implant. Optionally, the device includes a reservoir that maintains a stimulating drug. In this regard, the reservoir defines a delivery surface through which the drug is released from the reservoir. Finally, the reservoir is operatively associated with the electrode body to deliver the stimulating drug via the delivery surface to the brainstem or cerebellum following implant. During use, the electrode and the drug released from the reservoir act to simulate the brainstem or cerebellum, effecting cardiovascular regulation.

Hormonal or chemical (drug) agents function by interacting with specific receptor proteins on neurons. When activated by a neurotransmitter, hormone, or drug, these receptor proteins then either cause a chemical change in the cell, which indirectly causes ion channels embedded in the membrane to either open or close, thus causing a change in the electrical potential of the cell, or directly cause the opening of ion channels, which causes a change in the electrical potential of the cell.

Neural activity is constantly being controlled by the endogenous release of hormones, neurotransmitters, and neuromodulators. However, for therapeutic or experimental purposes, changes in neural activity can also be produced by the administration of chemical or hormonal agents (drugs) or incertain cases genetic material such as genes or messenger RNA. When administered exogenously, these agents interact with specific proteins either inside neurons or on the surface of the cell membrane to alter cell function. Chemical agents can stimulate the release of a neurotransmitter or family of neurotransmitters, block the release of neurotransmitters, block enzymatic breakdown of neurotransmitters, block reuptake of neurotransmitters, or produce any of a wide variety of other effects that alter nervous system functioning. A chemical agent can act directly to alter central nervous system functioning or it can act indirectly so that the effects of the drug are carried by neural messages to the brain. A number of chemical/hormonal agents such as epinephrine, amphetamine, ACTH, vasopressin, pentylene tetrazol, and hormone analogs all have been shown to modulate memory. Some act by directly stimulating brain structures. Others stimulate specific peripheral receptors.

In contrast, electrical stimulation of a nerve involves the direct depolarization of axons. When electrical current passes through an electrode placed in close proximity to a nerve, the axons are depolarized, and electrical signals travel along the nerve fibers. The intensity of stimulation will determine what portion of the axons are activated. A low-intensity stimulation will activate those axons that are most sensitive, i.e., those having the lowest threshold for the generation of action potentials. A more intense stimulus will activate a greater percentage of the axons.

Electrical stimulation of neural tissue involves the placement of electrodes inside or near nerve pathways or central nervous system structures. Functional nerve stimulation is a term often used to describe the application of electrical stimulation to nerve pathways in the peripheral nervous system. The term neural prostheses describes applications of nerve stimulation in which the electrical stimulation is used to replace or augment neural functions which have been damaged in some way. One of the earliest and most successful applications of electrical stimulation was the development of the cardiac pacemaker. More recent applications include the electrical stimulation of the auditory nerve to produce synthetic hearing in deaf patients, and the enhancement of breathing in patients with high-level spinal cord injury by stimulation of the phrenic nerve to produce contractions of diaphragm muscles. Recently, electrical stimulation of the vagus nerve has been used to attenuate epileptic seizures. In the present invention, it is preferable not to lesion any portion of the hindbrain and therefore electrodes which cause little or no physical damage to the medulla or hindbrain are preferred.

The basis of the effects of electrical stimulation of neural tissue comes from the observation that action potentials can be propagated by applying a rapidly changing electric field near excitable tissue such as nerve or muscle tissue. In this case, the electrical stimulation, when passed through an electrode placed in close proximity to a nerve or brain center, artificially depolarizes the cell membrane which contains ion channels capable of producing action potentials. Normally, such action potentials are initiated by the depolarization of a postsynaptic membrane. However, in the case of electrical stimulation, the action potentials are propagated from the point of stimulation along the axon to the intended target cells (orthodromic conduction). However, action potentials also travel from the point of nerve stimulation in the opposite direction as well (antidromic conduction).

One aspect of the present invention provides an improved neural stimulation device for treatment of cardiovascular disorders. The device includes an electrode body having an electrode implanted in or near the appropriate target sites in the cerebellum and brainstem and connected to an implantable pulse generator. The electrode body is configured for implantation within a patient so as to establish long-term contact between the electrode and the brainstem or cerebellum, the stimulation of which affects cardiovascular activity. Optionally, the device comprises a reservoir operatively associated with the electrode body. The reservoir maintains a stimulating drug. Further, the reservoir is configured to deliver the drug directly into the brainstem or cerebellum. Once delivered, the drug stimulates the brainstem or cerebellum, effecting an alteration in cardiovascular activity.

Yet another aspect of the present invention relates to a method for improved neural stimulation to treat cardiovascular disorders. The method includes stimulating the brainstem or cerebellum with an electrode. The nerve may be further stimulated with a stimulating drug delivered from a reservoir. In one preferred embodiment, delivery of the drug is correlated with activation of the electrode to generate an overall stimulation therapy. Current technology for both surface and depth electrode stimulation of the brain is commercially available and stimulation parameters can be extrapolated for the region of the brain involved. See U.S. Pat. No. 6,178,349 which is hereby incorporated by reference in its entirety.

To minimize electrical stimulation electrodes may remain off and only be turned on when sensor detects a cardiovascular condition out of control limits for blood pressure, heart rate, dissolved oxygen or other blood chemical indicating a cardiovascular condition including breathing rate. If a pH sensor is used on the lead, one such as that described in U.S. Pat. Nos. 4,009,721; 3,577,315; 3,658,053; or 3,710,778 may be used. A membrane pH sensor electrode is typically placed in the right ventricle and senses pH, which is proportional to the blood concentration of carbon dioxide, which in turn is generated in increasing amounts by exercise as explained in U.S. Pat. No. 4,716,887. In the '721 patent, a diminution in the pH level is used to produce a higher paced cardiac rate. However, if used in the context of the present invention, it is contemplated that the pH sensor will be placed on a lead just inside the coronary sinus to detect the level of lactic acid in venous return blood which is expected to increase with exercise of the cardiac muscle, particularly if the muscle is stressed by a lack of sufficient oxygen due to constriction in the cardiac arteries as a result of coronary artery disease. Myocardial ischemia is virtually invariably associated with an increase in the blood lactic acid level in the coronary sinus.

A dissolved blood oxygen sensor may be of the type described in Medtronic U.S. Pat. Nos. 4,750,495, 4,467,807 and 4,791,935. There, an optical detector is used to measure the mixed venous oxygen saturation.

The two neural mechanisms for controlling heart rate in the human and animal are the sympathetic and parasympathetic nervous systems. Sympathetic activity gives rise to relatively slowly varying changes in heart rate (e.g. below 0.1 Hz). Parasympathetic activity is generated in a region of the brain known as the Vital Centre, which is located in the lower medulla, and is transmitted to receptors in the sino-atrial node of the heart along the vagus nerve. The vagus nerve is myelinated such that parasympathetic activity is conveyed rapidly to the heart. The continuous flow of signal conveyed along the vagus nerve is termed the ‘vagal tone. Vagal tone tends to act as a ‘brake’ on the heart, slowing the heart rate to a lesser or greater extent. A high level of vagal tone also tends to give rise to relatively large and rapid fluctuations in heart rate period. Conventionally, it is these fluctuations which are used to measure vagal tone from recorded electrocardiograms (ECG) and to ‘isolate’ vagal tone from the relatively slowly varying effects of sympathetic activity. More particularly, vagal tone is generally measured by considering an ECG over a relatively long time period (e.g. 1000 beats) and evaluating the mean of the differences between consecutive beats. It is believed that certain diseases and conditions (e.g. diabetes and respiratory tract obstructions) can adversely effect cardiac function via parasympathetic activity. Vagal tone may be used for the purpose of monitoring, and possibly diagnosing, such diseases and conditions.

Immediate and future applications of the invention include direct surface stimulation of medullary cardiovascular centers, depth electrode stimulation of brainstem or cerebellar cardiovascular regulating centers, local extra-axial or intraparenchymal drug infusion into above-noted centers, and real-time close loop feedback system for each of the above wherein the hindbrain or brainstem therapeutic delivery device is regulated through blood pressure or heart rate feedback control sensor.

The apparatus and methods of this invention may be used for regulation and control of cardiovascular conditions including but not limited to essential hypertension, hypotension (Shy-Drager), paroxysmal atrial tachycardia, and bradycardia.

Although the invention has been described with reference to the preferred embodiments, it will be apparent to one skilled in the art that variations and modifications are contemplated within the spirit and scope of the invention. The drawings and description of the preferred embodiments are made by way of example rather than to limit the scope of the invention, and it is intended to cover within the spirit and scope of the invention all such changes and modifications.

What is claimed is:

1. An apparatus for modulating autonomic response in a vertebrate comprising: a therapeutic delivery device positioned near a site of the hindbrain structure of the vertebrate for modulating the function of the hindbrain and a controller in communication with the therapeutic delivery device to enable it to deliver the therapy.

2. The apparatus of claim 1 wherein said therapeutic delivery device is an electrode electrically connected to said controller.

3. The apparatus of claim 1 wherein said therapeutic delivery device delivers a pharmaceutical reagent to a site of said hindbrain structure and is connected to said controller.

4. The apparatus of claim 1 further comprising a sensor that measures a cardiovascular state of said vertebrate and is electrically connected to said controller.

5. The apparatus of claim 1 wherein said electrode is at a site near the surface of said hindbrain structure.

6. The apparatus of claim 1 wherein said electrode is implanted in the body of said vertebrate at a site near said hindbrain structure.

7. The apparatus of claim 6 wherein said hindbrain structure is selected from the group consisting of the medulla and the cerebellum.

8. The apparatus of claim 6 wherein said hindbrain structure is selected from the group consisting of the nucleus tractus solitarius, the caudal ventrolateral medulla, and the rostral ventrolateral medulla.

9. The apparatus of claim 1 wherein said electrodes are coated or comprise a composition that promotes adherence and growth of endogenous tissue and cells with said therapeutic delivery device to maintain the position of said therapeutic delivery device within said tissue.

10. The apparatus of claim 6 wherein said hindbrain structure chosen from the group consisting of fastigial nuclei and vestibular nuclei.

11. A method of controlling the cardiovascular state of a patient comprising:
comparing the cardiovascular state of a patient to a normal cardiovascular state and delivering a therapy from a therapeutic delivery device in a sufficient amount to a hindbrain structure to return the vertebrate to the normal cardiovascular state.

12. The method of claim 11 further comprising the step of measuring the cardiovascular state of the patient with sensors chosen from the group consisting of pH, blood pressure, heart rate dissolved oxygen, and dissolved carbon dioxide.

13. The method of claim 11 further comprising calculating the cardiac output.

14. The method of claim 11 wherein said therapy is electrical stimulation near a hindbrain structure.

15. The method of claim 11 wherein the steps of comparing the cardiovascular state and delivering the therapy to the patient are performed in a closed loop.

16. The method of claim 11 wherein the steps of comparing the cardiovascular state and delivering the therapy to the patient are performed in a closed loop using fuzzy logic rules.

17. The method of claim 11 wherein multiple therapeutic delivery devices are used and are enabled in response to the results of the step of comparing the cardiovascular state of the patient to a normal state.

18. The method of claim 11 wherein the step of delivering a therapy is changing the output from the therapeutic delivery device to the hindbrain structure, wherein the output from the therapeutic delivery device is chosen from the group consisting of voltage, pulse width, pulse frequency, current, drug delivery rate, and drug concentration.

19. The method of claim 11 wherein the therapy is a drug chosen from the group consisting of clonidine, guanethidine, a vetatrum alkaloid, and alpha-blockers, or specific neural excitatory or inhibitory transmitters and their antagonists such as gamma-aminobutyric acid (GABA), glycine, norepinephrine, acetylcholine (Ach), or nitric oxide (NO), proteins or enymes which modify the metabolism, release, binding and re-uptake of neurotransmitters, and genes and gene products which regulate cellular processes related to neural transmission.

20. The method of claim 11 wherein the cardiovascular condition is selected from the group consisting of essential hypertension, hypotension (Shy-Drager), paroxysmal atrial tachycardia, and bradycardia.
